(19) United States
(12) Patent Application Publication
(10) Pub. No.: US 2005/0215950 A1
(43) Pub. Date: Sep. 29, 2005

(54) Balloon catheter with radiopaque portion

(75) Inventors: Robert E. Burgmeier, Plymouth, MN (US); Richard Goodin, Blaine, MN (US); Angie Volk, Rogers, MN (US); Ray Adney, Coon Rapids, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/810,089

(22) Filed: Mar. 26, 2004

(51) Int. Cl. A61M 31/00

(52) U.S. Cl. 604/103.1

(57) ABSTRACT

A balloon catheter, and method of making the same, which includes an elongated shaft, and an expandable member affixed to the distal portion of the elongated shaft such that a section of the elongated shaft extends through at least a portion of the expandable member. The elongated shaft includes a radiopaque portion that includes a radiopaque material disposed in a non-metallic coating material. In some embodiments, the radiopaque portion is positioned adjacent the expandable member such that the position of at least a portion of the expandable member (or a stent disposed thereon) can be identified or determined within the vasculature in which it is deployed using an appropriate imaging technique, such as fluoroscopy. Additionally, in some embodiments, the radiopaque portion can define one or more raised areas on the elongated shaft adjacent the expandable member. In such embodiments, the raised area or areas in the radiopaque portion can function as mounting bodies for mounting a stent.

### FIELD OF THE INVENTION

This invention relates to the medical devices and more particularly to balloon catheters.

### BACKGROUND

Intravascular diseases are commonly treated by relatively non-invasive techniques such as percutaneous transluminal angioplasty (PTA) and percutaneous transluminal coronary angioplasty (PTCA). These therapeutic techniques are well known in the art and typically involve the use of a balloon catheter with a guidewire, possibly in combination with other intravascular devices such as stents. Some typical balloon catheters have an elongate shaft with a balloon attached proximate the distal end and a manifold attached to the proximal end. In use, some balloon catheters are advanced over a guidewire such that the balloon is positioned adjacent a restriction in a diseased vessel. The balloon is then inflated and the restriction in the vessel is opened.

Some basic types of intravascular catheters for use in such procedures, include, for example, fixed-wire (FW) catheters, over-the-wire (OTW) catheters and single-operator-exchange (SOE) catheters. The general construction and use of FW, OTW and SOE catheters are all well known in the art. An example of an OTW catheter may be found in commonly assigned U.S. Pat. No. 5,047,045 to Arney et al. An example of an SOE balloon catheter is disclosed in commonly assigned U.S. Pat. No. 5,156,594 to Keith.

Previous attempts to provide catheters that are more readily visualized within the vessel have involved the utilization of radiopaque marker members. A number of different catheter structures and assemblies are known, each having certain advantages and disadvantages. However, there is an ongoing need to provide alternative catheter structures and assemblies.

### SUMMARY

The invention provides several alternative designs, materials and methods of manufacturing alternative catheter structures and assemblies.

Some example embodiments provide a balloon catheter including an elongated shaft including a distal portion and defining at least one lumen. An expandable member is affixed to the distal portion of the elongated shaft such that a section of the elongated shaft extends through at least a portion of the expandable member. A radiopaque coating is disposed on a surface of a portion of the elongated shaft. The radiopaque coating includes a radiopaque material disposed within a non-metallic coating material that is applied to the surface of the segment of the shaft in a fluid state, and cured. In at least some embodiments, the coating is applied in an uncured or fluid state, and thereafter allowed to cure into a generally solid state.

In some example embodiments, a radiopaque portion is positioned adjacent the expandable member such that the position of at least a portion of the expandable member (or a stent or other such structure disposed thereon) can be identified or determined within the vasculature in which it is deployed using an appropriate imaging technique, such as fluoroscopy. Rendering the catheter identifiable proximate the expandable member can be helpful in guiding and positioning the catheter within the anatomy, for example, within the vasculature of a patient. For example, a radiopaque portion of a catheter can be viewed within body vasculature from outside the body to enable precise maneuvering and placement of the catheter with respect to a treatment area or to facilitate placement and deployment of a stent or other such structure, and the like.

In some embodiments, a radiopaque portion can define one or more raised areas on the tubular member adjacent the expandable member. In such embodiments, the raised area or areas in the radiopaque portion can function as mounting bodies for mounting another structure, for example, a stent. For example, the raised areas can provide a surface area or geometry of adequate diameter or size for mounting a stent, and the stent may be securely crimped upon the raised areas without exceeding the stent's minimum compression diameter.

Some other embodiments relate to methods of making and using balloon catheters.

The above summary of some embodiments is not intended to describe each disclosed embodiment or every implementation of the present invention. The Figures, and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a cross-sectional view of an example embodiment of a balloon catheter;

FIG. 2 is a partial cross-sectional view of an example embodiment of a distal portion of a balloon catheter for use on a catheter, for example, as in FIG. 1;

FIG. 3 is a partial cross-sectional view of another example embodiment of a distal portion of a balloon catheter for use on a catheter, for example, as in FIG. 1;

FIG. 4 is a partial cross-sectional view of another example embodiment of a distal portion of a balloon catheter for use on a catheter, for example, as in FIG. 1;

FIG. 5 is a partial cross-sectional view of another example embodiment of a distal portion of a balloon catheter for use on a catheter, for example, as in FIG. 1;

FIG. 6 is a partial cross-sectional view of the distal portion of a balloon catheter as in FIG. 5, including a stent mounted thereon; and

FIG. 7 is a partial cross-sectional view of another example embodiment of a distal portion of a balloon catheter for use on a catheter, for example, as in FIG. 1.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF SOME EMBODIMENTS

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description of some embodiments should be read with reference to the drawings, wherein like reference numerals indicate like elements throughout the several views. The drawings, which are not necessarily to scale, depict some example embodiments and are not intended to limit the scope of the invention. Those skilled in the art and others will recognize that many of the examples provided have suitable alternatives which may also be utilized.

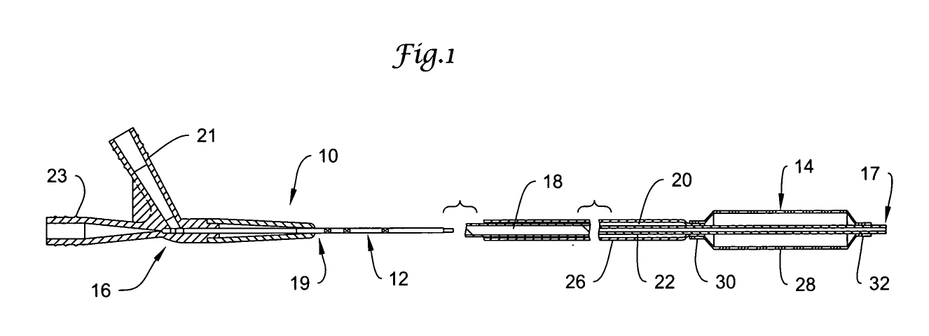

Referring now to the drawings, FIG. 1 is a cross-sectional view of an over-the-wire (OTW) balloon catheter 10, which is representative of one example type of catheter that can incorporate at least certain aspects of the invention. Other intravascular catheter embodiments are additionally suitable without deviating from the spirit and scope of the invention. For example, some other suitable intravascular catheters may include fixed-wire (FW) catheters, single-operator-exchange (SOE) catheters, and the like. Some examples of OTW catheters are disclosed in commonly assigned U.S. Pat. No. 5,047,045 to Arney et al., which is incorporated herein by reference. Some examples of SOE balloon catheters are disclosed in commonly assigned U.S. Pat. No. 5,156,594 to Keith, which is incorporated herein by reference.

The balloon catheter 10 can include a shaft assembly 12 and an expandable assembly, such as a balloon assembly 14, connected proximate the distal end of shaft assembly 12. The shaft assembly 12 may have conventional dimensions and may be made of conventional materials suitable for intravascular navigation as in, for example, conventional angioplasty, stent deployment procedures, or the like.

In some embodiments, the catheter shaft 12 comprises at least two lumens extending within the catheter shaft 12. At least one lumen can be a device and/or guidewire lumen 18 that is adapted and/or configured to receive a guidewire or other such medical device. In some embodiments, the lumen 18 may extend the entire length of the catheter shaft 12 (e.g. over-the-wire catheter), or it may extend along a portion of the catheter shaft 12, wherein it exits the catheter shaft 12 at the distal end 17 (e.g. single operator exchange catheter). The catheter shaft 12 can also include one or more additional lumens, for example, an inflation lumen 20. The inflation lumen 20, for example, may allow for fluid communication between an inflation source and the balloon assembly 14. In general, the proximal end of the inflation lumen 20 can be put into fluid communication with an inflation source while the distal end of the inflation lumen 20 is in fluid communication with the interior of the balloon assembly 14. The shaft assembly 12 may be a multiple lumen design or a coaxial design as shown.

In the co-axial design shown, the shaft assembly 12 can include an inner tubular member 22 and an outer tubular member 26. The inner tubular member 22 defines the guidewire lumen 18, and the outer tubular member 26 is co-axially disposed about the inner tubular member 22 to define the annular inflation lumen 20 there between.

In some embodiments, a manifold assembly 16 may be connected to the proximal end 19 of the shaft assembly 12. An example of a conventional OTW-type manifold assembly 16 is shown, but other types of manifolds are contemplated. In the example shown, one branch 21 of this manifold assembly 16 may be adapted and/or configured to connect an inflation source to the inflation lumen 20, and may be used to inflate and deflate an inflatable member 28. Another branch 23 of this manifold assembly 16 may connect to the guidewire lumen 18, and may be used for insertion of a guidewire or other such device into the lumen 18.

The balloon assembly 14 can include an expandable balloon portion 28, a proximal balloon waist 30 and a distal balloon waist 32. The proximal balloon waist 30 connects the balloon assembly 14 to the outer tubular member 26 near its distal end using suitable attachment means, for example, an adhesive, a thermal bond, a mechanical bond, or the like. The distal balloon waist 32 similarly connects the balloon assembly 14 to the inner tubular member 22 near its distal end using suitable attachment means, for example, an adhesive, a thermal bond, a mechanical bond, or the like. The inner tubular member 22 extends trough at least a portion of the expandable balloon portion 28 in a generally coaxial manner. In certain embodiments, the distal balloon waist 32 is only connected to the inner tubular member 22 which extends beyond the distal balloon waist 32. In alternative embodiments, the distal balloon waist 32 can be connected to the inner tubular member 22 and to a distal tip member (not shown) that extends distal of the inner tubular member 22 and the distal balloon waist 32.

Refer now to FIG. 2, which is an enlarged partial view of one example embodiment of a distal portion of a balloon catheter 10 similar in structure to that described above with reference to FIG. 1, wherein like reference numerals indicate similar structure. The catheter includes at least one radiopaque portion 40 that comprises a sheath or coating 41 made of a coating composition including non-metallic coating material having a radiopaque material disposed, loaded, embedded, or impregnated therein. The coating composition is applied and disposed on a surface of a segment of the inner tubular member 22 to form the coating 41. However, in other embodiments, depending upon the structure of the particular catheter, the coating composition may be applied and disposed on the surface of other structures of the catheter, for example, the outer tubular member 26, the balloon waists 30/32, or the like, to form the coating 41 thereon. The non-metallic coating material of the coating composition acts as a carrier for radiopaque material disposed therein.

The non-metallic coating material used in the coating 41 can include any material suitable for use as a coating disposed onto the desired surface, and that is appropriate for use as a carrier of the particular radiopaque material used. The coating material can include those that can be applied to the desired surface of the catheter 12 in a generally fluid an/or liquid state, and thereafter can transform or cure from the generally fluid an/or liquid state to a generally solid or semi-solid state on the surface to which they are applied.

For example, in some embodiments, the non-metallic coating material can include one or more of various paints, lacquers, varnishes, shellacs, resins, polymers, and the like, into which the desired radiopaque material can be disposed. Such coatings can applied using suitable application techniques, for example brushing, spraying, vapor deposition, electrostatic deposition, dip coating, extrusion, co-extrusion, interrupted layer co-extrusion (ILC), molding, casting, melting, forming, or the like, and other such techniques. The coatings can be cured using any suitable curing or drying method, depending upon the type of coating used. For example, some coatings can be air cured, heat cured, use a photoinitiated polymerization (e.g. visible, ultraviolet (U.V.), or infrared (I.R.) photoinitiated curing), or the like.

Some examples of suitable polymer coating materials can include thermoplastic polymers, appropriately modified thermosetting polymers, and the like. Some examples of suitable polymers include: polyether block amide (PEBA); polyethylene (for example, linear low and low density, as well as metallocene catalyzed varieties); polyethylene terephthalate (PET); polyurethane and polyurethane elastomers; polyphenylene sulfide (PPS); polyether sulfone (PES); polyesters in a variety of forms, including block co-polymers; polyamides; polyamide and polyester elastomers; polyethylene napthylate (PEN); polyimides; polycarbonate; polytrimethyl thalate (PTT); polyacetic acid (PLA); or co-polymers, mixtures or combinations thereof, as well as other, and the like. Some additional examples of suitable polymer materials include semi-compliant polyamides, or nylons, as well as hinged compliant materials such as polybutylene terephthalate (PBT) and Arnitel. Additionally, in some embobiments, the polymer can be blended with a liquid crystal polymer (LCP). For example, in some embodiments, a polymer mixture can contain up to about 6% LCP. This has been found to enhance torqueability.

In some embodiments, a coating polymer is used that can be disposed on the surface, and transformed from a liquid to a solid upon exposure to light, for example, ultraviolet light. Some examples of a photopolymerizable mixture working in accordance to a radical curing mechanism can comprise an unsaturated compound for film forming, and a photoinitiating system.

In some embodiments, the unsaturated compound for film forming can include an oligomer or a functional prepolymer sometimes called a resin exhibiting a molecular weight in the range of about 500-3000 and a viscosity in the range of about 5-25 Pa-s, that contains at least two reactive groups (vinyl, acetate, methacrylate , epoxy, etc.) and will constitute after polymerization the backbone of the polymer network. The physical as well as the chemical properties of the cured coating will depend of the nature and structure of the oligomer. Some example embodiments of such oligomers can be in accordance with the following formula:

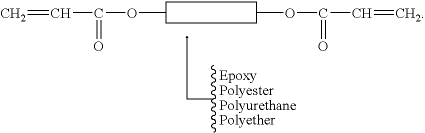

The unsaturated compound for film forming can also include a monomer that acts as a diluent to reduce the viscosity and thereby facilitate the handling of the formulation. This diluent can be reactive, for example, such that it readily participates in the polymerization process, and can contain several reactive groups. In at least some embodiments, the overall reaction is in the presence of a monofunctional monomer and oligomer, and may be considered as a kind of copolymerization whereas in the general case of interpenetrating networks is formed. Some examples of monomers that can be used in UV radical curing include: trimethylolpropane triacrylate (TMPTA); pentaerytrithol triacrylate (PETA); pentaerytrithol tri and tetra acrylate (PETIA); hydroxyethyl methacrylate (HEMA); hydroxyethylacrylate (HEA); ethyldiethyleneglycol acrylate (EDGA); hexanediol diacrylate (HDDA); tripropyleneglycol diacrylate (TPGDA); or the like, or others. Some additional examples of monomers that can be used include monofunctional monomers, such as Acticryl-SNPE, of the formula: 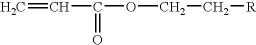
wherein R may be an oxazolidone; carbamate; carbonate; ether; ester; tetrafunctional monomers oligotetraacrylate; polyfunctional monomers (such as methacrylate diluting monomer), or the like, or others.

The photoinitiating system can include a photoinitiator and/or a mixture of photoinitiators and other initiators which produce free radicals and/or cations. Some examples of radical and cationic photoinitiators include: DEAP; DMPA; HCAP; TPMK; HAP; HAP derivative; C₁₂—HAP; titanocene derivative; morpholino ketone (BDMB); oligomeric HAP; trimethyl benzoyl phosphine oxide; hydrophilic HAP; 10-butyl-2-chloroacridone; 2-2-bis-(O-chlorophenyl)-4,5,4′,5′-tetraphenyl-1,2-bisimidazole; 4-benzoyl-4′-methyl diphenylsulfide; water-soluble thioxanthone; water-soluble copolymerizable benzophenone; 1-chloro,4-propoxy thioxanthone; ethyl p-dimethyl amino benzoate; 2-dimethylamino benzoate; 2-butoxyethyl-4-(dimethylamino)-benzoate; octyl p-dimethyl amino benzoate; amino acrylate; tri aryl sulfonium salt hexafluoro antimonite; bis[4-diphenylsulfonio)-phenyl]sulfid-bis-hexa fluorophosphates; iron-arene complex; di(alkylphenyl)iodonium salt, or the like, or others.

The radiopaque material disposed within the non-metallic coating material can include any material that when disposed within the non-metallic coating material can render the coating more visible when using certain imaging techniques, for example, fluoroscopy techniques. Some examples of radiopaque materials include, but are not limited to, gold, platinum, palladium, tantalum, tungsten, bismuth subcarbonate, and the like, or combinations, mixtures, or alloys of such materials. The radiopaque material is generally in a physical form that allows dispersal thereof within the non-metallic coating material. For example, the radiopaque material can be in a particulate form, such as powder, flakes, and the like, or combinations or mixtures thereof.

The radiopaque material can be present in the non-metallic coating material at the amount necessary to provide the desired radiopaque characteristics to the coating. In some embodiments, the radiopaque material can be present in the coating composition in the range of about 2 to about 95 wt. %, or in the range of about 80 to about 90 wt. %. In some embodiments, the particulate size of the radiopaque material can be controlled to achieve certain characteristics, for example, appropriate mixability of the radiopaque material with the non-metallic coating material, appropriate radiopaque characteristics, appropriate surface characteristics of the coating 41, or other such characteristics. In some embodiments, the radiopaque material has a particulate size in the range of about 1 Nanometer to about 100 μM, or in the range of about 1 to about 1000 Nanometers.

The coating 41 can be a single layer, or multiple layers of coating material. If multiple layer construction is used, one or more of the layers can include radiopaque material disposed therein.

In some embodiments, the outer surface of the radiopaque portion 40 is flush with the outer surface of other portions of the surface onto which it is coated. In some other embodiments, the outer surface of the radiopaque portion 40 is not flush with the outer surface of other portions of the inner tubular member 22 adjacent thereto, and can define, for example, a raised portion or a indented portion on the outer surface of the tubular member 22. For example, a raised portion 45 that extends radially outward from the outer surface of the elongated shaft is shown in FIG. 2. Such a raised portion 45 defined in the radiopaque portion 40 can aid in the mounting of another structure, such as a stent to the catheter, as will be discussed in more detail below.

The radiopaque portion 40 can be disposed at any desired location within the catheter, depending upon the desired visualization properties of the catheter. In the embodiment shown in FIG. 2, the radiopaque portion 40 is disposed adjacent the expandable balloon portion 28. The radiopaque portion 40 extends within the expandable balloon portion 28 from adjacent the proximal balloon cone X to adjacent the distal balloon con Y. Therefore, radiopaque portion 40 extends within the expandable balloon portion 28 for generally the entire length thereof. Generally non-radiopaque, or less radiopaque portions of the inner tubular member 22 extend both distally and proximally from the radiopaque portion 40. In other embodiments, the one or more radiopaque portions 40, each including a coating, could be disposed in alternative location adjacent the expandable balloon portion 28, or along other portions of the catheter. For example, one or more radiopaque portions 40 could be disposed on the surface of the inner and/or outer tubular member at a location spaced from the expandable balloon portion 28. Generally, the one or more radiopaque portions 40 are positioned such that the location of at least a portion of the expandable member 28, or another portion of the catheter 10, is identifiable or can be determined using a suitable imaging technique, for example, fluoroscopy. In the embodiment shown in FIG. 2, the radiopaque portion 40 is positioned such that the location of substantially the entire length of the expandable member 28 can be determined using a suitable imaging technique.

The method of making such a catheter 10 can include providing the shaft assembly 12, or portions and/or components thereof, and creating the radiopaque portion 40 at the desired locations on the desired surface of the shaft assembly 12 by applying the coating composition in a fluid state, and allowing it to cure. If necessary, further assembling of the shaft 12, or portions and/or components to create the catheter 10, may be performed. For example, in some embodiments, the inner and outer tubular members 22/26 can be provided, a suitable radiopaque coating composition can be applied to a section of the inner tubular member where desired and allowed to cure to create the coating 41. The tubular members 22/26 can be assembled to create the shaft 12, and any additional structures and assemblies, such as the balloon assembly 14, a manifold 16, and the like, can be also incorporated to form the catheter 10.

Refer now to FIG. 3, which shows another alternative embodiment of a distal portion of a balloon catheter 10 similar in structure to that described above with reference to FIG. 1, wherein like reference numerals indicate similar structure. In the embodiment shown in FIG. 3, the inner tubular member 22 includes two radiopaque portions 140 and 142 located under or within the expandable member 28. The radiopaque portions 140 and 142 can be created using a radiopaque coating composition as discussed above. The radiopaque portions 140 and 142 are positioned such that the location of at least a portion of the expandable member 28 is identifiable or can be determined using a suitable imaging technique, for example, fluoroscopy. The radiopaque portion 140 is positioned adjacent the proximal end of the expandable member 28, and the radiopaque portion 142 is positioned adjacent the distal end of the expandable member 28. As such, the ends of the expandable member 28 can be identified, and therefore the location of the entire length of the expandable member 28 can be determined using a suitable imaging technique. Each of the radiopaque portions 140 and 142 can include a single layer, or multiple layers, and can be disposed and/or attached to the surface of the inner tubular member 22 using any suitable technique for the particular material used and to achieve the configuration or pattern desired. Additionally, as discussed with regard to the embodiments shown in FIG. 2, the outer surface of the radiopaque portions 140 or 142 could be flush, or could define raised or indented portions on the outer surface of the tubular member 22. In FIG. 3, raised portions 145 and 147 are shown that extend radially outward from the outer surface of the elongated shaft. Such raised portions 145 and 147 may aid in the mounting of another structure, for example, a stent to the catheter, as will be discussed in more detail below.

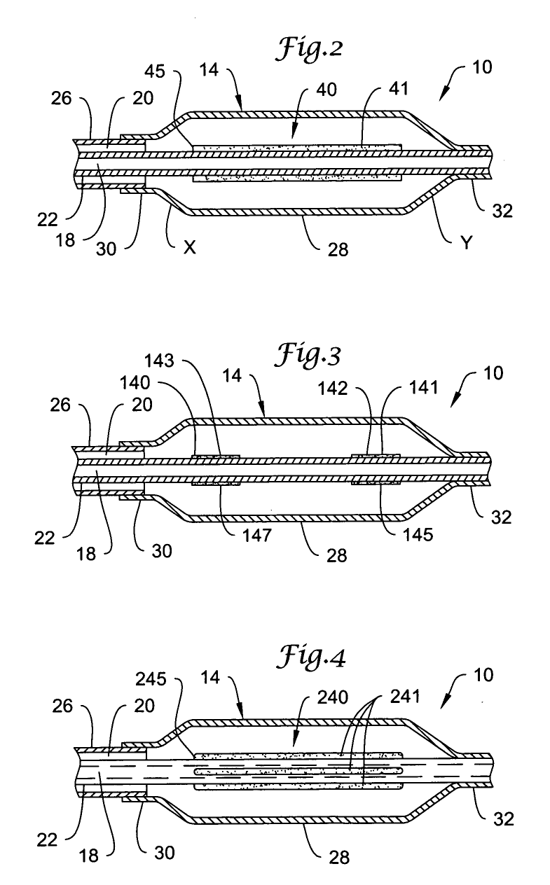

Refer now to FIG. 4, which shows another alternative embodiment of a distal portion of a balloon catheter 10 similar in structure to that described above with reference to FIG. 1, wherein like reference numerals indicate similar structure. In the embodiment of FIG. 4, the catheter includes at least one radiopaque portion 240 that comprises one or more segments 241 disposed on the outer surface of the tubular member 22, wherein each of the segments 241 comprises a non-metallic coating material having a radiopaque material disposed, loaded, embedded, or impregnated therein, for example as discussed above. The radiopaque segments 241 can be created using a radiopaque coating composition as discussed above. Each of the segments 241 can be oriented in any desirable position to give a desired pattern or radiopaque signal. In the embodiment shown, four elongated segments 241 (three are shown) are disposed in a generally linear and parallel configuration relative to one another about the longitudinal axis of the inner tubular member 22. In other embodiments, more or fewer segments 241 can be used, for example 1, 2, 3, 5, 10, 20 or more such segments 241 may be used. The segments 241 can be oriented in any desirable configuration. For example, the segments 241 can be arranged in configurations such as in a helical arrangement, a grid arrangement, annular rings, diagonal lines, and the like, on the surface of the inner tubular member 22. As discussed above, each of the segments 241 can include a single layer, or multiple layers, and can be disposed and/or attached to the surface of the inner tubular member 22 using any suitable technique for the particular coating material used and to achieve the configuration or pattern desired. Again, in some embodiments, the one or more segments 241 can define one or more raised portions 245 that extend radially outward from the outer surface of the elongated shaft on the outer surface of the tubular member 22, as shown. Such raised portions can aid in the mounting of another structure, for example, a stent to the catheter, as will be discussed in more detail below.

Refer now to FIG. 5, which shows another alternative embodiment of a distal portion of a balloon catheter 10 similar in structure to that described above with reference to FIG. 1, wherein like reference numerals indicate similar structure. In the embodiment of FIG. 5, the catheter includes at least one radiopaque portion 340 that comprises one or more segment 341 disposed on the outer surface of the tubular member 22. The segment 341 is similar to the segments 241 discussed above with reference to FIG. 4, but is disposed about the inner tubular member 22 in a generally helical fashion. The radiopaque segment 341 can be created using a radiopaque coating composition as discussed above. Again, in some embodiments, the segment 341 can define one or more raised portions 345 on the outer surface of the tubular member 22 that extend radially outward from the outer surface of the elongated shaft, as shown. Such raised portions can aid in the mounting of another structure, for example a stent, to the catheter, as will be discussed in more detail below.

As discussed above with regard to various embodiments, in some embodiments, the radiopaque portion or portions can define one or more raised portions on the outer surface of the inner tubular member 22. These raised portions can aid in the mounting of another structure, such as a stent to the catheter 10. Stents and stent delivery assemblies are utilized in conjunction with vascular angioplasty. Because dilated stenoses are known to reobstruct, a stent is often implanted to maintain the patency of the vessel.

A stent is a generally cylindrical prosthesis which is introduced, for example, via a balloon catheter, into a lumen of a body vessel. The stent is positioned, and secured onto, the balloon in a configuration having a generally reduced diameter. Once the balloon catheter is positioned adjacent the desired location within the vasculature, the balloon is expanded. This balloon expansion subsequently causes the stent to increase its radial configuration from a reduced diameter (delivery diameter) to an expanded one (deployment diameter). In its expanded configuration, the stent supports and reinforces the vessel wall while maintaining the vessel in an open and unobstructed configuration.

The structure and functions of stents are well known. Some examples of stents used in conjunction with vascular angioplasty are shown in U.S. Pat. No. 5,064,435 to Porter; U.S. Pat. No. 5,071,407 to Termin et al.; U.S. Pat. No. 5,221,261 to Termin et al.; U.S. Pat. No. 5,234,457 to Anderson; U.S. Pat. No. 5,370,691 to Samson; U.S. Pat. No. 5,378,239 to Termin et al.; U.S. Pat. No. 5,401,257 to Chevalier, Jr. et al.; and U.S. Pat. No. 5,464,450 to Buschemi et al., all of which disclosures are incorporated herein by reference.

A distinguishable feature between stents is whether they are self-expanding or balloon expandable. Both self-expanding and balloon expandable stent are well known and widely available. Certain embodiments of catheters incorporating certain embodiments of the invention relate to enhanced stent securement and loading in the delivery and deployment of balloon expandable stents.

Balloon expandable stents are crimped to their reduced diameter about the balloon portion of the catheter assembly. The stents are gently crimped onto the balloon either by hand, or with a tool. Once the stent is mounted, the catheter system is ready for delivery. There are, however, two complications associated with crimping stents to balloon catheters: (1) excessive crimping may damage the stent, the balloon, or the inner lumen of the catheter; and (2) inadequate securement force results in failure of the stent to maintain its axial position during advancement within the human anatomy.

Most expandable stents have a minimum compression diameter. The minimum compression diameter is the smallest radial profile that a stent may be reduced to without causing damage to the stent. This damage often decreases the functionality and reliability of the stent's expansion, as well as its ability to maintain the patency of a vessel wall. Furthermore, the stent must be crimped over that portion of the balloon which is expandable in order to have the entire length of the stent expanded against the vessel wall on deployment. The expandable portion balloons in some cases have an insufficient outer diameter for direct attachment of a stent in the balloon's folded, deflated configuration. Therefore, crimping a stent on this section alone will cause the stent to bend undesirably or it will not be held adequately in axial position without artificially building-up the diameter under the balloon—or other means to create bulk for stent crimping.

Some embodiments of catheters including radiopaque portions as described above that define one or more raised portions, for example raised portions 45, 145, 147, 245, and 345 shown in FIGS. 2-5, on the shaft 12, for example on the outer surface of the tubular member 22, can serve as a means to create bulk for stent crimping. In some embodiments, the raised portions can serve as mounting bodies that are disposed on the surface of the inner tubular member 22 under the expandable balloon portion 28. These raised portions extend radially from the inner tubular member 22, and can provide a surface area of adequate diameter for mounting a stent. A stent, therefore, may be securely crimped or otherwise disposed upon the raised portions without exceeding the stent's minimum compression diameter.

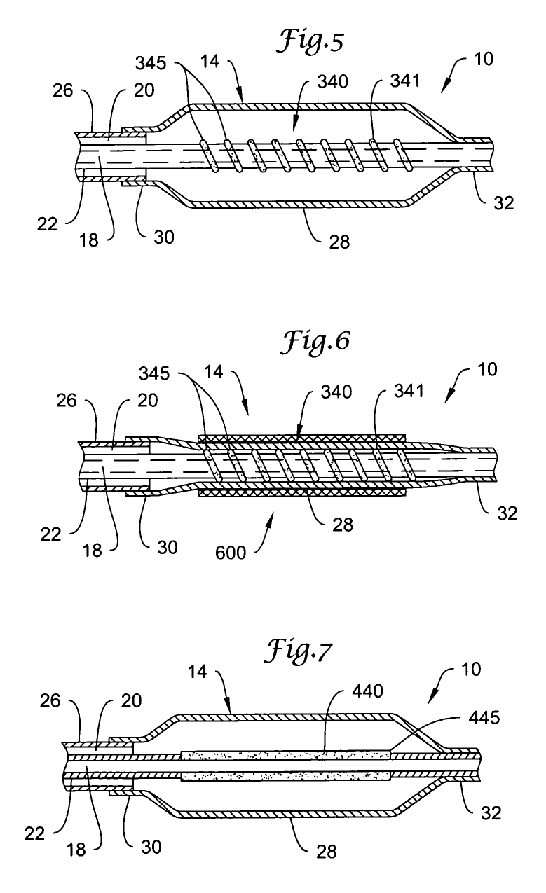

For example, refer now to FIG. 6, which shows the distal portion of a balloon catheter 10 of FIG. 5, wherein like reference numerals indicate similar structure, including a stent 600 mounted about the expandable member 28. In this embodiment, the one or more segment 341 of the radiopaque portion define one or more raised portions 345 on the outer surface of the tubular member 22, as shown. The one or more raised portions 345 on the outer surface of the tubular member 22 can act as mounting bodies or structures that, for example, can aid in mounting the stent to the balloon catheter. For example, the raised portions can provide a cushion and/or substrate of enlarged diameter relative to the stent 600 to aid in supporting and/or holding the stent during and/or after crimping and/or during a delivery procedure. The one or more raised portions 345 can aide in preventing excessive crimping of the stent, the balloon, or the inner lumen of the catheter; and can aide in ensuring adequate securement force resulting in the stent maintaining its axial position during advancement within the human anatomy. Some additional disclosure related to attaching an expandable stent to a stent delivery device is provided in U.S. Pat. No. 6,203,558, which is incorporated herein by reference.

In some other embodiments, such radiopaque raised portions can be achieved using other structure. For example, refer now to FIG. 7, which shows another alternative embodiment of a distal portion of a balloon catheter 10 similar in structure to that described above with reference to FIG. 1, wherein like reference numerals indicate similar structure. This embodiment, however, includes a raised portion 445 that extends radially outward from the outer surface of the elongated shaft, and that is defined by a radiopaque portion 440 that is part of the shaft 12 itself rather than a coating applied to the surface of a part of the shaft 12. The shaft 12 may include and/or be made of a non-metallic material, and include the raised portion 445 defined in its shape. The raised portion may include a radiopaque material disposed, loaded, embedded, or impregnated within the non-metallic material thereof. In other words, a portion of the shaft 12 can define the raised portion 445 and include a non-metallic material including a radiopaque material disposed therein that forms the raised portion 445. In the embodiment shown, the raised portion 445 is defined by a portion of the tubular member 22, and the raised portion 445 includes the non-metallic material of the inner tubular member 22 that can be loaded with radiopaque material. The non-metallic material of the inner tubular member 22 acts as a carrier for the radiopaque material.

In at least some embodiments, the raised radiopaque portion 445 can be described as being a portion of, integral with, or of unitary or monolithic construction with the remainder of the inner tubular member 22. The raised portion 40 and other portions of the inner tubular member 22 can include or be made of the same or different material, for example the polymer materials and/or radiopaque materials discussed above, and each can include additional suitable materials or combinations of materials to achieve the desired structure and characteristics for the inner tubular member 22.

The inner tubular member 22 including the raised radiopaque portion 445 can be formed using any suitable technique to achieve the desired structure. For example, techniques such as extrusion, co-extrusion, interrupted layer co-extrusion (ILC), molding, casting, forming, grinding, thermal bonding, shrink bonding, adhesives bonding, welding, mechanical bonding, or the like, can be used to form the tubular member 22 including the raised radiopaque portion 445.

In some other embodiments, the raised radiopaque portion 445 can be independently formed and thereafter attached to the shaft 12, for example, attached to a portion of the inner member 22. Again, the raised radiopaque portion 445 can be made of a non-metallic material including a radiopaque material disposed, loaded, embedded, or impregnated within the non-metallic material thereof. For example, the raised radiopaque portion 445 may be an polymeric annular or tubular member including a radiopaque material disposed therein. Attachment the raised radiopaque portion 445 to the shaft 12 can be accomplished through using suitable attachment techniques, for example, thermal bonding, adhesives bonding, shrink bonding, mechanical connection, material welding, or other suitable attachment techniques. Once disposed on the shaft, the annular or tubular member would define the radiopaque raised portion 445 that extends radially outward from the outer surface of the elongated shaft, and that is defined by a radiopaque portion 440 that is a separate member attached to the shaft rather than being part of the shaft, or a coating applied to the surface of a part of the shaft 12.

Having thus described some embodiments of the invention, those of skill in the art will readily appreciate that yet other embodiments may be made and used within the scope of the claims hereto attached.

1. A balloon catheter comprising:
an elongated shaft including a distal portion and defining at least one lumen;
an expandable member affixed to the distal portion of the elongated shaft such that a section of the elongated shaft extends through at least a portion of the expandable member; and
a radiopaque coating disposed on a surface of a portion of the elongated shaft, the radiopaque coating comprising a radiopaque material disposed within a non-metallic coating material that is applied to the surface of the segment of the shaft in a fluid state and cured.

2. The catheter of claim 1, wherein the radiopaque coating is disposed adjacent the expandable member

3. The catheter of claim 1, wherein the non-metallic coating material comprises paint, lacquer, varnish, shellac, or resin.

4. The catheter of claim 1, wherein the non-metallic coating material comprises non-metallic polymer coating material loaded with the radiopaque material.

5. The catheter of claim 1, wherein the non-metallic coating material comprises a coating material that can be cured through photoinitiated polymerization.

6. The catheter of claim 1, wherein the radiopaque coating provides one or more raised portions that extend radially from the outer surface of the elongated shaft to provide one or more mounting structures for use in mounting a stent to the catheter.

7. The catheter of claim 1, wherein the radiopaque coating extends through at least a portion of the expandable member.

8. The catheter of claim 1, wherein the entire radiopaque coating is disposed within the expandable member.

9. The catheter of claim 1, wherein the elongated shaft includes two or more radiopaque coatings, each radiopaque coating comprising a radiopaque material disposed within the non-metallic coating material that is applied to a surface of a portion of the shaft in a fluid state and cured.

10. The catheter of claim 1, wherein the radiopaque coating is a first radiopaque coating, and the catheter further includes a second radiopaque coating;
the expandable member includes a proximal end and a distal end; and
the first radiopaque coating is disposed proximate the proximal end of the expandable member, and the second radiopaque coating is disposed proximate the distal end of the expandable member.

11. The catheter of claim 10, wherein the radiopaque coatings define raised portions that extend radially from the elongated shaft and provide a surface area of adequate diameter for mounting a stent.

12. The catheter of claim 1, wherein the radiopaque coating includes one or more separate segments of radiopaque coating disposed on the surface of the portion of the shaft.

13. The catheter of claim 12, wherein the radiopaque coating includes in the range or 1 to 6 separate segments of radiopaque coating disposed on the surface of the portion of the shaft.

14. The catheter of claim 12, wherein the one or more segments are disposed in a helical arrangement about the shaft.

15. The catheter of claim 12, wherein the one or more segments are disposed in a longitudinal arrangement along a longitudinal axis of the shaft.

16. The catheter of claim 12, wherein the one or more segments are disposed in a grid arrangement

17. A balloon catheter comprising:
an outer tubular member having a proximal end and a distal end, and defining at least one lumen;
an inner tubular member having a proximal end and a distal end, and including an outer surface and defining at least one lumen, the inner tubular member at least partially coaxially disposed within the lumen of the outer tubular member to form an inflation lumen there between;
an inflatable balloon having a proximal end sealably connected proximate the distal end of the outer tube, and a distal end sealably connected proximate the distal end of the inner tube, an interior of the balloon being in fluid communication with the inflation lumen; and
a radiopaque coating disposed on a portion of the surface of inner tubular member adjacent the inflatable balloon, the radiopaque coating comprising a radiopaque material disposed within a non-metallic coating material that is applied to the surface of the segment of the portion of the inner tubular member in a fluid state and cured.

18. The balloon catheter of claim 17, wherein at least a portion of the radiopaque coating is disposed on a portion of the inner tubular member that extends through at least a portion of the inflatable balloon.

19. The balloon catheter of claim 17, wherein the catheter includes two or more separate radiopaque coatings disposed on the surface of inner tubular member.

20. The balloon catheter of claim 17, wherein the radiopaque coating defines one or more raised portions that extend radially from the inner tube and provides a surface area of adequate diameter for mounting a stent.

21. A balloon catheter comprising:
an elongate outer tube having a lumen extending the length therein;
an elongate inner tube coaxially disposed within at least a portion of the elongate outer tube with a distal segment extending distally beyond a distal end of the elongate outer tube, the elongate inner tube having at least a portion being radiopaque by coating the portion of the inner tube with a non-metallic coating material loaded with a radiopaque material where the non-metallic coating material is applied to the surface of the segment of the shaft in a fluid state and cured; and
an inflatable balloon having a proximal end, a distal end and an expandable region therebetween, wherein the distal end of the balloon is sealably connected to the distal segment of the elongate inner tube and the proximal end of the balloon is sealably connected to the elongate outer tube such that the expandable region of the balloon is in fluid communication with the lumen of the elongate outer tube, and
wherein the radiopaque portion of the inner tube is positioned adjacent the inflatable balloon.

22. A balloon catheter comprising:
a tubular member including a distal portion;
an expandable member affixed to the distal portion of the tubular member such that a portion of the tubular member extends through at least a portion of the expandable member; and
coating means for rendering a portion of the tubular member adjacent the expandable member identifiable under fluoroscopy, wherein the coating means is applied in a fluid state and cured.

23. A balloon catheter comprising:
an elongated shaft including a distal portion and defining at least one lumen;
an expandable member affixed to the distal portion of the elongated shaft such that a section of the elongated shaft extends through at least a portion of the expandable member; and
a radiopaque portion disposed on a surface of a segment of the elongated shaft, the radiopaque portion comprising a radiopaque material disposed within a non-metallic material, the radiopaque portion defining one or more raised portions that extend radially from the elongated shaft and provide a surface area of adequate diameter for mounting a stent, the radiopaque portion applied to the surface of the segment of the shaft in a fluid state and cured.

24. A balloon catheter produced by a process comprising:
providing an elongated shaft including a distal portion and defining at least one lumen;
applying a radiopaque coating in a fluid state to a surface of a portion of the elongated shaft, the radiopaque coating comprising a radiopaque material disposed within a non-metallic coating material;
allowing the radiopaque coating to cure; and
affixing an expandable member to the distal portion of the elongated shaft such that a section of the elongated shaft extends through at least a portion of the expandable member.

25. A method of making a balloon catheter, the method comprising:
providing an elongated shaft including a distal portion and defining at least one lumen;
applying a radiopaque coating in a fluid state to a surface of a portion of the elongated shaft, the radiopaque coating comprising a radiopaque material disposed within a non-metallic coating material;
allowing the radiopaque coating to cure; and
affixing an expandable member to the distal portion of the elongated shaft such that a section of the elongated shaft extends through at least a portion of the expandable member.

26. The method of claim 25, wherein the expandable member is affixed to the shaft adjacent the radiopaque coating.

27. The method of claim 25, wherein the non-metallic coating material comprises paint, lacquer, varnish, shellac, or resin.

28. The method of claim 25, wherein the non-metallic coating material comprises non-metallic polymer coating material loaded with the radiopaque material.

29. The method of claim 25, wherein the non-metallic coating material comprises a coating material that can be cured through photoinitiated polymerization, and allowing the radiopaque coating to cure includes curing the coating material through photoinitiated polymerization.

30. The method of claim 25, wherein applying a radiopaque coating includes applying a sufficient amount of the radiopaque coating to the surface such that upon curing, the coating provides one or more raised portions that extend radially from the outer surface of the elongated shaft to provide one or more mounting structures for use in mounting a stent to the catheter.

31. The method of claim 25, wherein the radiopaque coating extends through at least a portion of the expandable member.

32. The method of claim 25, wherein the entire radiopaque coating is disposed within the expandable member.

33. The method of claim 25, wherein two or more radiopaque coatings are applied to the elongated shaft, each radiopaque coating comprising a radiopaque material disposed within the non-metallic coating material that is applied to a surface of a portion of the shaft in a fluid state and allowed to cured.

34. The method of claim 25, wherein applying a radiopaque coating includes applying the radiopaque coating in one or more separate segments on the surface of the portion of the shaft.

35. The method of claim 34, wherein applying a radiopaque coating includes applying in the range of 1 to 6 separate segments of radiopaque coating on the surface of the portion of the shaft.

36. The method of claim 34, wherein applying a radiopaque coating includes applying the one or more segments in a helical arrangement about the shaft.

37. The method of claim 34, wherein the one or more segments are applied in a longitudinal arrangement along a longitudinal axis of the shaft.

38. The method of claim 34, wherein the one or more segments are applied in a grid arrangement onto the shaft.

39. A method of making a balloon catheter, the method comprising:
providing an outer tube having a proximal end and a distal end with a lumen extending there through;
forming an inner tube having a proximal end and a distal end with a lumen extending there through, wherein the inner tube includes a radiopaque portion comprising a radiopaque material disposed in a non-metallic carrier;
disposing at least a portion of the inner tube within at least a portion of the outer tube such that at least a portions of the tubes are in a coaxial arrangement and define an inflation lumen there between; and
affixing an inflatable balloon proximate the distal end of the outer tube, and proximate the distal end of the inner tube such that the radiopaque portion is adjacent the balloon, and the interior of the balloon is in fluid communication with the inflation lumen.

40. A method of making a balloon catheter, the method comprising:
providing an elongated shaft including a distal portion and defining at least one lumen;
providing the elongates shaft with a radiopaque portion disposed on a surface of a segment of the elongated shaft, the radiopaque portion comprising a radiopaque material disposed within a non-metallic material, the radiopaque portion defining one or more raised portions that extend radially from the elongated shaft and provide a surface area of adequate diameter for mounting a stent; and
affixing an expandable member to the distal portion of the elongated shaft such that a section of the elongated shaft extends through at least a portion of the expandable member.
